(19) United States
(12) Patent Application Publication
(10) Pub. No.: US 2004/0038213 A1
(43) Pub. Date: Feb. 26, 2004

(54) Genotyping by in situ PCR amplification of a polynucleotide in a tissue biopsy

(76) Inventor: Jai W. Kwon, South Pasadena, CA (US)

Correspondence Address: PARK & SUTTON LLP, 3255 WILSHIRE BLVD, SUITE 1110, LOS ANGELES, CA, 90010, US

(21) Appl. No.: 10/213,009

(22) Filed: Aug. 6, 2002

(51) Int. Cl. C12Q 1/68; C12P 19/34; C12N 1/08

(52) U.S. Cl. 435/6; 435/91.2; 435/270

(57) ABSTRACT

Reagents and method for genotyping mice and other animals by in situ Polymerase Chain Reaction amplification of a target polynucleotide in the tissue biopsy. The reagent is comprised of non-ionic detergents, a protease, a buffering agent, a metal ion cofactor, a chelating agent and a salt. The method is comprised of taking a tissue biopsy; admixing it with the reagent; a Lysing Cycle, an inactivation cycle, an amplification step and a detection step.

### CROSS-REFERENCE TO RELATED APPLICATIONS

None

### STATEMENT REGARDING FEDERALLY SPONSORED RESEARCH OR DEVELOPMENT

None

### REFERENCE TO MICROFICHE APPENDIX

None

### BACKGROUND OF THE INVENTION

1. Field of the Invention

This invention pertains generally to the art of releasing polynucleotides from cells and more particularly to genotyping through in situ Polymerase Chain Reaction amplification of a target gene allele in a tissue biopsy.

2. Related Art

A new mouse strain can be derived using technologies known as targeted mutagenesis and site directed mutagenesis (see, B. Hogan et al., “Manipulating The Mouse Embryo: A Laboratory Manual,” (Cold Spring Harbor Laboratory Press, N.Y.) (1994) (incorporated by reference).) These technologies are used to make additions in or removal of one or more gene alleles. The mouse is described as “genetically altered.” Where a gene is added, the mouse is referred to as “transgenic.” Where the change is to remove a gene, the mouse is described as a “knock out” mouse (see, E. M. Simpson, “Genetic Variation Among 129 substrates And Its Importance For Targeted Mutagenesis In Mice,” Nature Genetics 16: 19-27 (1997) (incorporated by reference).)

Genetically altered mice have been used to study gene function and human disease. Over the past decade, thousands of new mouse strains have been developed which will eventually cover all of the genes in a mouse. Genetically altered mice have provided researchers and the medical community with an immense new resource of biological tools and information. Morris Alpert called the development of knock out mice the most significant advance in research of the twentieth century.

Sonya Swing of Taconic Transgenics (Germantown, N.Y.) writes that, “Scores of novel transgenic mouse lines are being produced each year, and as the human and mouse genome projects continue to generate data, that number is expected to escalate. The number of transgenic models that are becoming standard research tools is also increasing because of the study of the role of specific genes in human disease. Transgenic mouse models are used in virtually every area of biomedical research, including carcinogenicity, drug testing, and therapeutic approaches such as gene therapy. Consequently, transgenic models are finding their way into all types of research laboratories, from academic to government and pharmaceutical laboratories.”

A research study typically involves lots of 500 genetically altered mice and each mouse needs to be genotyped. In genotyping, a tail biopsy is commonly used as a source of chromosomes (DNA). To facilitate detecting a gene allele in the biopsy, it is common practice to amplify a portion of the chromosome containing the gene allele using Polymerase Chain Reaction amplification (see, R. K. Saiki et al., “Primer-directed Enzymatic Amplification of DNA With A Thermostable DNA Polymerase,” Science, pp. 487-491 (1988) (incorporated by reference).) The amplified fragment is then detected by methods such as hybridization to a polynucleotide array on a slide, a dot blot, a Southern blot, and the like.

In the tail biopsy, the chromosomes are located inside cells. Each of the cells is surrounded by a cell membrane. Within the cell, the chromosomes are found inside an internal structure known as nuclear envelope. The chromosomes are complexed and associated with proteins, such as histones.

In addition to chromosomes, the cells in the tail biopsy contains an estimated 100,000 chemicals and biomolecules. Amongst these chemical and biomolecules are nucleases, proteases and inhibitors. These nucleases, proteases and inhibitors are inimical to Polymerase Chain Reaction amplification of DNA.

The tail biopsy also contains body components other than cells. The other body components are such things as hair, skin, ligaments, cartilage, blood vessels, blood and the like. The mouse tail biopsy is typically reddish in color, due blood components. These other body components interfere with extraction of DNA and Polymerase Chain Reaction amplification.

There are three common methods for carryout the genotyping utilizing tail biopsies. These methods are typically called the “Phenol/chloroform method,” “spooling method” and “DNeasy kit method.”

The Phenol/chloroform method is known from F. Ausubel et al., “Current Protocols in Molecular Biology,” (Wiley Interscience, N.Y.) (2000) (incorporated by reference).) It uses tail biopsies that are typically 0.6 cm in the length. The cut tails are solubilized using a multicomponent lysising solution. There is a first preliminary clean step; namely, centrifuging to settle down hairs and insoluble materials. After centrifugation, phenol/chloroform solution is added as denaturant of soluble proteins. There is a second set of preliminary clean up and DNA isolation steps; namely, a phase separation; followed by ethanol precipitation; followed by centrifugation; followed by aspiration; followed by washing with ethanol; followed by centrifugation; followed by drying and resolubolizing. Aliquots can then be taken for the PCR amplification. This method has the disadvantages of requiring numerous and expensive reagents; being tedious and time consuming and requiring preliminary clean up steps.

The Spooling method is known from P. W. Aaird et al., “Simplified Mammalian DNA Isolation Procedure,” Nucleic Acids Research, 19:4923 (1991) (incorporated by reference).) It uses tail biopsies that are typically 0.6 cm in the length. The cut tails are solubilized using a multicomponent lysising solution. There is a first preliminary clean step; namely, centrifuging to settle down hairs and insoluble materials which is then followed by washing with isopropanol. After washing, there is a DNA isolation step, namely, chromosomes are aggregated using a sample loading tip and the aggregated chromosomes are spooled out of solution. There is a second set of preliminary clean up steps; namely, removing excess solutions by scraping and attaching the aggregates onto the dry surface of a tip; followed by second scraping and attaching onto a new tip; followed by solubilizing and then incubating. Aliquots can then be taken for the PCR amplification. This method has the disadvantages of requiring numerous and expensive reagents and supplies; being tedious and time consuming and requiring preliminary clean up steps and DNA isolation.

The DNeasy kit method is a product by Qiagen, Inc. (Valencia, Calif.) called “The DNeasy 96 Tissue Kit.” It is described in the Qiagen Product Guide, pp. 97-103 (1999) (incorporated by reference).) The method is for DNA isolation from rodent tails and other tissues. Tail or tissue samples are lysed. There is a set of preliminary clean up and DNA isolation steps using silica-gel-membrane technology for isolation of total cellular DNA; namely, buffering conditions are carefully adjusted to an essential and proper pH; followed by centrifuging; followed by binding of genomic DNA to a column; followed centrifuging using a DNeasy spin column or 96-well plate; followed by washing and rewashing and followed by elution. Aliquots can then be taken for the PCR amplification. This method has the disadvantages of requiring numerous and very expensive reagents and supplies; being tedious and time consuming and requiring preliminary clean up steps and DNA isolation.

It is known in the art from U.S. Pat. No. 5,543,305 (incorporated by reference) to use non-ionic detergents as part of a composition to extract nucleic acids from whole cells from animal sources for amplification by Polymerase Chain Reaction amplification (“PCR”). The method is described as for use with fresh or frozen cell pellets. Before PCR can be performed, a critical step of the method is that cellular matter and coagulated debris be separated from the fluid containing soluble DNA by filtration, centrifugation, decanting or siphoning. This method has the disadvantages of requiring isolated whole cells not including other body components such as hair, skin, ligaments, cartilage, blood vessels, blood and the like. It also has the disadvantage of requiring clean up before performing PCR.

It is known in the art from U.S. Pat. No. 6,242,188 (incorporated by reference) to use a composition containing a non-ionic detergent to extract nucleic acids from whole cells from animal sources for in situ amplification by Polymerase Chain Reaction amplification (“PCR”). The method is described as for use with whole cells obtained from body fluids or in solid tissue samples. The composition is comprised of many chemicals, in addition to non-ionic detergent including, inter alia, lipid, organic solvent, ionic surfactant, proteinase and EDTA. The disclosure teaches the use of ionic surfactant (detergent) to neutralize proteases. This method has the disadvantages of requiring isolated whole cells not including other body components such as hair, skin, ligaments, cartilage, blood vessels, blood and the like. The method also has the disadvantage of requiring a complex and costly composition with uncertainty as to what is to be included and not included so that the composition is not inhibitory of the polymerase in the PCR step.

Accordingly, there exists a need for a method for Polymerase Chain Reaction amplification using crude lysates of mouse tails without DNA isolation.

There exists a need for a method to genotype a mutant mouse without DNA isolation.

There exists a need for a method and reagents to reduce the time, cost and complexity of obtaining polynucleotides from cells in an animal tissue biopsy in a form suitable for In Situ Polymerase Chain Reaction amplification.

The present invention satisfies these needs, as well as others, and generally overcomes the presently known deficiencies in the art.

### SUMMARY OF THE INVENTION

The present invention is directed to methods for genotyping without DNA isolation and to reagents for use in the methods. In various aspects of the invention, non-ionic detergents are used for lysing tail biopsies to allow for the direct use of crude lysates for Polymerase Chain Reaction.

An object of the present invention is to provide methods and reagents to avoid tedious and costly DNA isolation steps for genotyping using mouse tails.

Another object of the present invention is to provide a reagent kit useful for genotyping mutant mice.

One aspect of the present invention is a composition for releasing polynucleotides from an animal tissue biopsy comprised of cells having polynucleotides along with other body components in a form suitable for in situ Polymerase Chain Reaction of a target polynucleotide without preliminary clean up steps. The composition is comprised of the components described as follows. One component is a combination of two or three non-ionic detergents selected from the group consisting of short chain octylphenoxy polyethoxy ethanol, medium chain octylphenoxy polyethoxy ethanol, long chain octylphenoxy polyethoxy ethanol, short chain polyoxyethlene sorbitan monolaurate, medium chain polyoxyethlene sorbitan monolaurate and long chain polyoxyethlene sorbitan monolaurate, each of the selected detergents at a concentration such that the combination is effective to facilitate the release polynucleotides from cells in the animal tissue biopsy.

Another component is a protease at a concentration that is effective to facilitate the release polynucleotides from the cells with the polynucleotides being in a condition that they are sufficiently free from nucleic acid associated proteins such that a nucleic acid amplification step can be performed. Another component is a buffering agent which buffers the lysing reagent at a pH which is conducive to the functioning of both the protease and a polynucleotide polymerase to be added in an amplification step.

Another component is a metal ion cofactor at a concentration that is effective to activate the protease and that is ineffective to significantly deactivate a polynucleotide polymerase to be added in an amplification step. Another component is a chelating agent at a concentration that is effective to sufficiently inactivate attacking agents in the tail biopsy and that is ineffective to significantly chelate the metal ion cofactor. Another component is a salt at a concentration effective to approximate physiological conditions for both the protease and a polynucleotide polymerase to be added in an amplification step.

Another aspect of the present invention is a method for genotyping an animal. The method is comprised of the steps describes as follows. The first step is taking an animal tissue biopsy comprised of cells having polynucleotides along with other body components. The second step is forming an admixture of the animal tissue biopsy with an effective amount of the composition described above. The third step is heating the admixture for a period of time and under conditions effective to lyse a sufficient quantity of cells in the animal tissue biopsy, so as to form a crude lysate.

The fourth step is heating the crude lysate for a period of time and under conditions such that the protease and attacking agents in the animal tissue biopsy are significantly inactivated, so as to form a lysate. The fifth step is amplifying in the lysate a target polynucleotide using a polynucleotide polymerase in an amplification method, without preliminary cleanup steps. The sixth step is detecting the target polynucleotide sequence whereby a genotype of the animal is determined.

Another aspect of the present invention is a kit for genotyping a animal by taking a biopsy and detecting a target nucleic acid sequence through in situ Polymerase Chain Reaction without preliminary clean up steps. The kit is comprised of a vial containing the composition as described above and one or more vials containing nucleic acid probes or primers complementary to the polynucleotide to be detected.

The previously described versions of the present invention has many advantages which include saving time; saving money; eliminating the use of phenol/chloroform which is dangerous to humans and the environment; minimizing the use of plasticware, such as tubes and tips, which are harmful to the environment; reducing the consumption of proteinase K; reducing loss of DNA from a tissue biopsy and reducing pain to rodents.

### BRIEF DESCRIPTION OF THE DRAWINGS

These and other features, aspects and advantages of the present invention will become better understood with reference to the following description, appended claims and accompanying drawings where:

FIG. 1 is a photograph (image) of an electrophoresis gel illustrating the detection of amplified polynucleotides from mice tail biopsies derived according to the present invention and in particular, the detection of a 618 base pair (bp) DNA fragment from the Y chromosome and a 221 bp DNA fragment from the X chromosome for gender determination of mice and

FIG. 2 is a photograph (image) of an electrophoresis gel illustrating the detection of amplified polynucleotides from mice tail biopsies derived according to the present invention and in particular, the detection of a 409 base pair (bp) DNA fragment from the Y chromosome and a 221 bp DNA fragment from the X chromosome for gender determination of mice.

### DETAILED DESCRIPTION OF THE INVENTION

The present invention provides methods and reagents to genotype a mutant mouse without DNA isolation. In aspects of the present invention, mouse tails are lysed by a reagent containing non-ionic detergents to form a crude lysate. The crude lysates are treated with heat and directly used for Polymerase Chain Reaction without DNA isolation. The methods and reagents of this invention save time and costs.

One aspect of the present invention are compositions for releasing polynucleotides from biopsies of animal tissue comprised of cells having polynucleotides along with other body components in a form suitable for in situ Polymerase Chain Reaction amplification of a target polynucleotide without preliminary clean up steps comprised of a lysing reagent. An interrelated aspect of the invention are compositions for releasing polynucleotides from biopsies of a mouse's tail comprised of cells having polynucleotides along with other body components in a form suitable for in situ Polymerase Chain Reaction amplification of a target polynucleotide without preliminary clean up steps comprised of a lysing reagent. Other aspects of the present invention are compositions for releasing a polynucleotides where the polynucleotides are chromosomes or chromosome fragments and the target polynucleotide is a portion of the chromosome or chromosome fragment. The portion usually being a gene allele.

The lysing reagent includes a combination of at least two or three non-ionic detergents (surfactants). The believed purpose is to permeabilize the nuclear envelope and cell membrane so as to facilitate, at least in-part, the release of polynucleic acids (and in particular, chromosomes or chromosome fragments) from inside the nuclear envelope and cytoplasmic membrane of the cell. This permeablization may be accomplished by fluidizing and dissolving, part of or all of, the nuclear envelope and cell membrane. The detergents function without adversely affecting the polynucleic acids (and in particular, chromosomes or chromosome fragments).

A preferred combination of two or three non-ionic detergents is selected from the group: NP40™ brand octylphenoxy polyethoxy ethanol sold by Sigma-Aldrich (St. Louis, Mo.), Triton X-100™ brand octylphenoxy polyethoxy ethanol sold by Rohm & Haas (Philadelphia, Pa.) and Tween-20™ brand polyoxyethlene sorbitan monolaurate sold by Imperial Chemical Industries Americas, Inc. (Bridgewater, N.J.) The most preferred combination is all three of the forgoing.

Those skilled in the art will recognize that protocols can be developed, without undue experimentation, for particular applications using non-ionic detergents having properties equivalent to or substitutable for the branded non-ionic detergents described above.

Similarly, those skilled in the art will recognize that protocols can be developed, without undue experimentation, for particular applications using non-ionic detergents selected from the group consisting of short chain octylphenoxy polyethoxy ethanol, medium chain octylphenoxy polyethoxy ethanol, long chain octylphenoxy polyethoxy ethanol, short chain polyoxyethlene sorbitan monolaurate, medium chain polyoxyethlene sorbitan monolaurate and long chain polyoxyethlene sorbitan monolaurate.

Similarly, those skilled in the art will recognize that protocols can be developed, without undue experimentation, for particular applications using non-ionic detergents where one or more of the nonionic detergents is selected from the following group of branded non-ionic detergents: polyoxyethylene ether sold under the brand name TRITON X-102, nonylphenoxy polyethoxy ethanol sold under the brand name Triton-N, polyoxyethylene (4) sorbitan monolaurate sold under the brand name Tween-21, polyoxyethylene (20) sorbitan monopalmitate sold under the brand name Tween-40, polyoxyethylene (20) sorbitan monostearate sold under the brand name Tween-60 polyoxyethylene (23) lauryl ether sold under the brandname Brij-35 by Imperial Chemical Industries Americas (see above) and polyoxyethylene (2) cetyl ether sold under the brand name Brij-52. These non-ionic detergents are regarded as having properties equivalent to or substitutable for the branded trademarks discussed above in particular applications.

Similarly, those skilled in the art will recognize that protocols can be developed, without undue experimentation, for particular applications where one or more of the non-ionic detergents is selected from the group consisting of 3-(2-aminopropyl-1,3-dihexadecyloxypropyl) hexadecyl ether, 3-(2aminopropyl-1-octadecyloxy-3-benzyloxypropyl) benzyl sulfide and bis(3-benzyloxypropyl-1-octadecyloxy-3-benzyloxy-2-propyl amine)-polyethyleneglycol. These non-ionic detergents are regarded as having properties equivalent to or substitutable for the branded trademarks discussed above in particular applications.

The concentrations of the non-ionic detergents are set based on the consideration that each be present at a concentration such that the combination of non-ionic detergents is effective to facilitate the release polynucleotides (and in particular chromosomes and chromosome fragments) from cells in the animal tissue biopsy (and in particular, a mouse tail biopsy.) A preferred concentration is that the two or three nonionic detergents in the combination each be present at a nonzero concentration that sums to about 0.6% (v/v).

Using NP40, Triton X-100 and Tween-20 as the combination of non-ionic detergents, a most preferred and believed to be optimal concentrations of the nonionic detergents providing superior results is 0.1% NP40, 0.1% Triton X-100 and 0.4% Tween-20. The next most preferred concentrations are 0.2% NP40, 0.2% Triton X-100 and 0.2% Tween-20. The next most preferred concentrations are 0.1% NP40, 0.4% Triton X-100 and 0.1% Tween-20. The next most preferred concentrations are 0.4% NP40, 0.1% Triton X-100 and 0.1% Tween-20. The next most preferred concentrations are 0.3% Triton X-100 and 0.3% Tween-20.

The lysing reagent includes a protease. The believed purpose of the protease is to digest proteins associated with polynucleic acids (and in particular, chromosomes or chromosome fragments) so as to facilitate the polynucleic acids being released from the cells. For example and more specifically, histones associated with chromosomes. The protease cuts and/or degrades the polynucleic acid associated proteins into smaller peptides sequences and individual amino acids. By so doing the polynucleic acids, and in particular, chromosomes and chromosome fragments, become more soluble in solution.

A preferred protease is proteinase K. Those skilled in the art will recognize that protocols can be developed, without undue experimentation, for particular applications using a protease other than or in conjunction with proteinase K. For example, in some applications any one or combination of the following may be effective and appropriate: trypsin, chymotrypsin and V8 protease. Current commercial sources for proteinase K are Sigma-Aldrich Corp. (St. Louis, Mo.) and Fisher Scientific, Inc. (Hampton, N.H.)

The concentration of the protease is set based on four considerations. One consideration is that concentration be large enough so as to be effective to release polynucleotides (chromosomes) from the cells. Second, that the concentration be large enough that the polynucleotides (chromosomes) be in a condition of being sufficiently free from polynucleic acid associated proteins such that a polynucleic acid amplification step can be effectively performed. Third, that the concentration of the protease be small enough so that after a Heat Inactivation Cycle (explained below) it does not to significantly attack a polynucleotide polymerase to be added in an amplification step (see below). A significant attack occurs when the polynucleotide polymerase cannot effectively function in an amplification step. Fourth, the lower the concentration of protease, the less attack on the polynucleotide polymerase; but, the longer the needed time for heating the crude lysate in a Lysing Cycle (explained below) and the shorter the period of time for heating in an Heat Inactivation Cycle (explained below.)

Using proteinase K as the protease and Taq (thermus acquaticus) as the polynucleotide polymerase, the concentration of proteinase K is typically between about 0.1 mg/ml to about 0.5 mg/ml. A preferred concentration is about 0.3 mg/ml.

The lysing reagent includes a buffer that maintains the pH of the lysing reagent at a particular level. The believed purpose of the buffer is to maintain the pH level of lysing reagent at level that is conducive to the functioning of the protease and a polynucleotide polymerase.

A preferred buffer is Tris-HCl. Those skilled in the art will recognize that protocols can be developed, without undue experimentation, for particular applications using a buffer other than or in conjunction with Tris-HCl. For example, in some applications any one or combination of the following may be effective and appropriate: 3-(N-morpholino)propanesulfonic acid, 3-(N-morpholino)ethanesulfonic acid, tricine, glycine, tris(hydroxymethyl)aminomethane and HEPES (4-(2-Hydroxyethyl)-1-piperazineethane-sulfonic acid). Current commercial sources for the buffer agent are Sigma-Aldrich Corp. (St. Louis, Mo.) and Fisher Scientific, Inc. (Hampton, N.H.)

The concentration of the buffer is set based on a consideration that it be such that the buffer maintains the lysing reagent at a pH level that is a compromise between the optimal pH for the functioning of the protease and the polynucleotide polymerase.

Using proteinase K as the protease and Taq (thermus acquaticus) as the polynucleotide polymerase, the buffer typically maintains the pH of the lysing reagent between about 8.3 to about 8.8. More preferably, the buffer maintains the pH between about 8.5 to about 8.8. Most preferably, the buffer maintains the pH of the lying reagent at about 8.5. Those skilled in the art will recognize that protocols can be developed, without undue experimentation, for particular applications where the buffer maintains the pH outside the typical range. In the development of such protocols, it is unlikely that a pH below about 8.0 will be functional pH for the lysing reagent.

The typical buffering range of about 8.3 to about 8.8 is achieved by concentrations of Tris-HCl between about 50 mM to about 150 mM in an aliquot of 200 microliters of lysing reagent. At lower concentrations, capacity of the buffer will be lost. At higher concentrations, the buffer will interfere with the functioning of the proteinase K and Taq. The most preferred pH of about 8.5 is achieved by a concentration of Tris-HCl of about 100 mM in an aliquot of 200 microliters of lysing reagent.

The lysing reagent includes a suitable metal ion cofactor, also referred to as an enhancer. The believed purpose of the metal ion cofactor is to activate (i.e., enhance) the protease. Typically, the metal ion cofactor is a bivalent metal cation. The metal ion cofactor can be supplied as a free ion or as a salt.

A preferred source for a metal ion cofactor is MgCl₂. A nonrecommended metal cofactor is Ca²⁺. Calcium is not recommended because, it functions as a precipitator and stabilizer. One of the believed purposes for including a chelating agent (discussed below) in the lysing reagent is to chelate out Ca²⁺ which activates endogenous attacking agents that are released from the cells other tail components in the biopsy of the mouse's tail (explained further below.) Those skilled in the art will recognize that protocols can be developed, without undue without undue experimentation, for particular applications using a metal ion cofactor other than or in conjunction with MgCl₂. For example, in some applications one or combination of the following may be effective and appropriate: magnesium acetate, magnesium bromide, magnesium sulfate, manganese chloride and/or manganese bromide. Current commercial source for the metal ion cofactor is Sigma-Aldrich Corp. (St. Louis, Mo.) and Fisher Scientific, Inc. (Hampton, N.H.)

The concentration of the metal ion cofactor is set a level based on two considerations. One consideration is that the concentration be large enough that there is sufficient metal ion cofactor to activate (enhance) the protease. The other consideration is the metal ion cofactor concentration be small enough so as not to poison the polynucleotide polymerase. Typically, a polynucleotide polymerase is sensitive metal ion cofactor concentration.

Using Mg²⁺ as the metal ion cofactor, proteinase K as the protease and Taq (thermus acquaticus) as the polynucleotide polymerase, typically there is a ratio of Mg²⁺ to proteinase K of about 1:1 to about 1:2. Concentrations of Mg²⁺ above 5 mM interfere with and/or poison the Taq. The typical concentration range of Mg²⁺ is from about 1.0 mM to about 5.0 mM. A sometimes preferred concentration is about 1.5 mM. A most preferred concentration is about 3 mM.

The lysing reagent includes a chelating agent. The believed purpose of the chelating agent is to have an inactivating effect on endogenous nucleases, proteases and inhibitors in the biopsy of animal tissue (and in particular a mouse's tail) that are released from cells and other components. These endogenous nucleases, proteases and inhibitors are collectively referred to herein as “attacking agents.” The chelating agent has an inactivating effect on attacking agents by chelating out bivalent metal ions that are endogenous to the cells and other components that activate these attacking agents. As explained above, one such bivalent metal ion is Ca⁺⁺.

Typically, the chelating agent is ethylenediaminetetraacetic acid (EDTA) and ethyleneguaninetetraacetic acid (EGTA) or combination of the two. A preferred chelating agent is ethylenediaminetetraacetic acid (EDTA). Those skilled in the art will recognize that protocols can be developed, without undue experimentation, for particular applications using a chelating agent other than or in conjunction with EDTA or EGTA. For example, in some applications it might be effective and appropriate to use bis-picolylamine. Current commercial sources for the chelating agent are Sigma-Aldrich Corp. (St. Louis, Mo.) and Fisher Scientific, Inc. (Hampton, N.H.)

The concentration of the chelating agent is set a level based on two considerations. One consideration is that the concentration be large enough that there be a sufficient amount of chelating agent to accomplish the believed purpose of having an inactivating effect on attacking agents. The amounts needs to be sufficiently large so that in conjunction with de-activation for a Heating Inactivation cycle (discussed below) polymerase chain reaction amplification can be effectively performed. The other consideration is that the concentration be small enough so as not to significantly chelate the metal ion cofactor.

Using EDTA as the chelating agent and MgCl₂ as the metal ion cofactor, the typical concentration range is from about 0.5 mM to about 2 mM. A concentration below about 0.5 mM is not sufficient to accomplish the inactivation function and a concentration above about 2 mM chelates out the MgCl₂. A preferred concentration is about 1.0 mM.

The lysing reagent includes a salt. The believed purpose of the salt is to approximate physiological conditions such that the protease and polynucleotide polymerase function efficiently.

Typically, the salt is NaCl and KCl. A preferred salt is NaCl. Calcium salts are not recommended because, as explained above, Ca²⁺ functions as a precipitator and stabilizer and activates endogenous attacking agents that are released from the cells tail components in the biopsy. Those skilled in the art will recognize that protocols can be developed, without undue experimentation, for particular applications using other salts; for example, salts of monovalent cations that are non-toxic to enzymes. Current commercial sources for the salt are Sigma-Aldrich Corp. (St. Louis, Mo.) and Fisher Scientific, Inc. (Hampton, N.H.)

The concentration of the salt is set based on the consideration of approximating physiological conditions so as to optimize the function of the protease and polynucleotide polymerase. Typically, the concentration is between about 50 mM to about 200 mM. A preferred concentration is about 150 mM.

Another aspect of the present invention are methods to genotype an animal by taking a tissue biopsy comprised of cells having polynucleotides along with other components body components and using Polymerase Chain Reaction amplification to amplify a target nucleotide without preliminary clean up steps. An interrelated aspect of the present invention are methods to genotype a mouse by taking a biopsy of the mouse's tail cells having polynucleotides along with other components body components and using Polymerase Chain Reaction amplification to amplify a target nucleotide without preliminary clean up steps. Other aspects of the present invention are methods to genotype an animal, and in particular, a mouse, where the polynucleotides are chromosomes or chromosome fragments and the target polynucleotide is a portion of the chromosome or chromosome fragment. The portion of the chromosome usually being a gene allele.

The animal being biopsied is any animal used in research. Besides mice, such animals include, by way of example, rats, guinea pigs, other rodents, rabbits, monkeys, sheep and the like.

The first step of the method is to take an animal tissue biopsy comprised of cells having polynucleotides. The biopsy is preferably a tail section. Where tails are unavailable, or not desirable, the biopsy can be from any portion of the body (preferably, consistent with ethical and humane treatment of animals) or the animal can be sacrificed. In taking the biopsy it is permissible to include other body components such as hair, skin, ligaments, cartilage, blood vessels, blood and the like. Standard procedures for taking a biopsy known to those skilled in the art are used.

Using a mouse and taking a biopsy of the mouse tail, the biopsy is typically from about 0.1 cm to about 0.6 cm in length, exclusive of hair. In terms of mass, the biopsy is typically between about 0.1 mg to about 0.6 mg. The preferred biopsy length is about 0.6 cm, exclusive of hair. Likewise, in terms of mass, the preferred biopsy is about 0.6 mg. The lower limit of about 0.1 cm (exclusive of hair) is based on feasible cutting technology. It is envisioned that cutting technology will improve and shorter biopsy lengths will be feasible.

The mouse tail biopsy is typically reddish in color, due blood components. As stated above, it includes other body components, such as, hair, skin, ligaments, cartilage, blood vessels, blood and the like. It also includes an estimated 100,000 chemicals and biomolecules. The chemical and biomolecules include attacking agents. As specified above, “attacking agents” are endogenous nucleases, proteases and inhibitors.

The next step is forming an admixture of the animal tissue biopsy with an effective amount of lysing reagent. An effective amount of lysing reagents is determined based on three considerations. A first consideration is the amount of animal tissue biopsy. The more animal tissue, the more lysing reagent that is needed. Subject to other considerations (discussed below), the amount of lysing reagent is directly proportional to amount of animal tissue biopsy. Second the length of a Lysing Cycle (described below) and a Heat Inactivation Cycle (described below). During these cycles, there is evaporation. In an extreme case, the evaporation could result in a drying out of all liquid. Generally, a longer Lysing Cycle requires more lysing reagent. In such circumstances, it may be preferable to make a two fold or more dilution of the lysing reagent. The dilutions are made using additional buffer. Keeping in mind the considerations stated above, it may be necessary to proportionately increase the amount of protease so that its concentration remains the same after dilution. A third consideration is that there be a sufficient volume of lysing reagent to engulf the tissue biopsy and allow for mixing between the tissue biopsy and lysing reagent during the Lysing Cycle (described below.)

Using mouse tails as the animal tissue biopsy, and the preferred biopsy length of about 0.6 cm (as stated above) the preferred amount of lysing reagent is about 200 microliters. For an about 0.3 cm long mouse tail biopsy, the preferred amount of lysing reagent is about 100 microliters. For an about 0.1 cm long mouse tail biopsy, the preferred amount of lysing reagent is about 40 microliters of an about 2× diluted lysing reagent. If only about 20 microliters of lysing reagent were used, it might evaporate off in its entirety in the Lysing Cycle (described below) and there would not be a sufficient volume to engulf and mix with the tissue biopsy during the Lysing Cycle. In making the about two-fold dilution, about twice the amount of buffer is used along with about twice the amount of protease to keep its concentration about the same after dilution.

A vessel in which to perform the admixing are well known to those skilled in the art. Usually, the same vessel is used in a Lysing Cycle (described below.) There are four considerations in selecting a vessel. First, the size of the vessel be comparable to the size of the tissue biopsy and volume of lysing reagent such that the lysing reagent engulfs the animal tissue biopsy and there be mixing between the animal tissue biopsy and the lysing reagent. Second, that the shape of the lysing vessel be such that the lysing reagent engulfs the animal tissue biopsy and there be mixing between the animal tissue biopsy and the lysing reagent. Third the vessel be non-reactive to the lysing reagent and not bleed into the lysing reagent any significant quantity of plasticizers or other agents so as to be toxic to the protease and/or a polynucleotide polymerase (mentioned above and discussed below.) Fourth, the vessel be heat stable and conducting at the temperatures at which the Lysing Cycle is performed, if it also be used in the Lysing Cycle.

Using between about 0.3 cm to about 0.6 cm mouse tails as the animal tissue biopsy, it is preferred that the vessel be a 1.7 ml microcentrifuge tube. Using between about 0.1 cm to about 0.29 cm mouse tails as the animal tissue biopsy, it is preferred that the vessel be a 0.75 ml microcentrifuge tube. A current commercial source for microcentrifuge tubes is Brinkmann Instruments, Inc. a division of Eppendorf Company (Westbury, N.Y.).

The next step is heating the admixture for a period time and under conditions effective to lyse a sufficient quantity of cells in the animal tissue biopsy so as to form a crude lysate. This step is sometimes referred to herein as the “lysing cycle.” A sufficient quantity of cells are lysed when there is enough chromosomes (or polynucleotide) available that contain the target polynucleotide (a portion of a gene allele) to be amplified such that Polymerase Chain Reaction can be conducted (discussed below.) There are three main variables regarding the conditions for heating the admixture. These variables are temperature, time and heating environment.

The temperature is set based on three considerations. The first consideration is that the temperature be high enough that nuclear envelope and cell membrane are permeabilize; i.e., that they are fluidized and/or dissolved in whole or in part. Second, that the temperature be conducive to the functioning of the protease. Third that the temperature not be so great that the Brown Movement (vibration) of the chromosome (DNA) increases that there is significant thermal degradation. Using mouse tails as the animal tissue biopsy and proteinase K as the protease, the preferred temperature is between about 50° C. to about 55° C. A temperature of about 60° C. will usually be ineffective.

The time is set based on two considerations. The first consideration is the size of the animal tissue biopsy. The greater the size of the animal tissue biopsy, the longer the Lysing Cycle. The second consideration is the amount of protease. The greater the amount of protease, the shorter the Lysing Cycle time. The Lysing Cycle is sensitive to the minimum time. It is usually not that sensitive to the maximum time. Accordingly, for convenience, the lysing can be allowed to run overnight. The typical Lysing Cycle is about 4 to about 8 hours long.

Using mouse tails as the animal tissue biopsy and proteinase K as the protease, the table below presents minimal lysing times.

| Proteinase K | | | |
|---|---|---|---|
| concentration\ | | | |
| Tail size | 0.6 cm | 0.3 cm | 0.1 cm |
| 0.1 mg/ml | 8 hrs | 5 hrs | 3 hrs |
| 0.3 mg/ml | 3 hrs | 2 hrs | 1.5 hrs |
| 0.5 mg/ml | 2.5 hrs | 1.5 hrs | 1.2 hrs |

The environment are those known to individuals in the art that can provide a stable temperature within the considerations set forth above and mixing for the animal tissue biopsy with the lysing reagent. A preferred environment is a rotating hybridization oven. The oven has roller bed. Onto this roller be is seated from about 1 to about 6 glass tubes. Each of the glass tubes holds about 25 microcentrifuge tubes. A current commercial source for a rotating hybridization oven is Hybrid Oven Systems of Sasib UK Ltd. (Merseyside, Whales.) A current commercial source for the glass tube is BD (Beckton Dickinson) (Franklin Lakes, N.J.)

The next step is heating the crude lysate for a period time and under conditions such that the protease and attacking agents in the animal tissue biopsy are significantly inactivated so as to form a lysate. This step is sometimes referred to herein as the “Heat Inactivation cycle” or “Inactivation cycle.” The protease and attacking agents in the animal tissue biopsy are significantly inactivated when their level of activity is low enough that Polymerase Chain Reaction can be effectively conducted (discussed below.) There are three main variables regarding the conditions for heating the crude lysate. These variables are temperature, time and heating environment.

The temperature is set based on three considerations. The first consideration is that the temperature be high enough to significantly inactivate the protease. If the protease is not significantly inactivated, it will attack the polynucleotide polymerase of the Polymerase Chain Reaction (discussed below.) The second consideration is that the temperature be high enough to significantly inactivate the attacking agents. As discussed above, the animal tissue biopsy includes an estimated 100,000 chemicals and biomolecules. The chemical and biomolecules include attacking agents such as nucleases, proteases and inhibitors. Likewise, if the attacking agents are not significantly inactivated, they will attack the polynucleotide polymerase of the Polymerase Chain Reaction (discussed below.)

The third consideration is that the temperature be sufficiently below the melting temperature of the DNA (chromosomes.) As is well known since Watson & Crick, a chromosome is a helical and double stranded DNA polynucleotide. The melting point of DNA is defined as the temperature at which an average of 50% of the base pairs are detached from being double stranded. The result being two single strands. The DNA is vibrating and there is a dynamic as to which bases are detached. DNA generally melts at 92° C.

Single stranded DNA is more prone to shearing, breaking and/or fragmenting. The temperature of the Heat Inactivation Cycle is set below the melting temperature of DNA at a level that does not result in significant shearing, breaking and/or fragmenting of chromosomes. Significant shearing, breaking and/or fragmenting occurs when it results in insufficient chromosomes containing the target polynucleotide to conduct PCR amplification and subsequent detection of the target polynucleotide.

Using mouse tails as the animal biopsy, a preferred temperature for the Heat Inactivation Cycle is about 85° C. A less preferred temperature is between more than about 85° C. and less than about 90° C. In this less preferred temperature range, there is more single stranded DNA than at the preferred temperature. The temperature of about 90° C. is about 2° C. below the general melting point of DNA. Temperatures higher than about 90° C. are disfavored.

The time for the Heat Inactivation Cycle is determined based on two considerations. The first consideration is that the time be long enough to significantly inactivate the protease and attacking agents by denaturation and thermal degradation. As stated above, if the proteases and attacking agents are not significantly inactivated, they will attack the polynucleotide polymerase of the Polymerase Chain Reaction (discussed below.) The second consideration is that the time be short enough that significant shearing, breaking and/or fragmenting of chromosomes does not occur. The longer the Heat Inactivation Cycle, the more single stranded DNA that is transiently or permanently present. Commensurately, the greater the opportunities for shearing, breaking and fragmenting.

Using mouse tails as the animal tissue biopsy and proteinase K as the protease, the Heat Inactivation Cycle lasts from between about 30 minutes to about 3.0 hours. At times shorter than about 30 minutes, there will be insufficient deactivation of proteinase K and at times longer than 3.0 hours there will be too much single stranded DNA that is vulnerable to breaking. A most preferred time for the Heat Inactivation Cycle is about 45 minutes.

The next step in the method is amplifying in the lysate a target polynucleotide using a polynucleotide polymerase in an amplification method. Polymerase Chain Reaction is the preferred amplification method. Persons of ordinary skill in the art will recognize that in certain applications, other methods may be suitable. These other methods include ligase chain reaction (LCR), transcription mediated amplification (TMA) reaction, nucleic acid sequence based amplification (NASBA) reaction, and strand displacement amplification (SDA) reaction. These methods of amplification are known in the art. LCR can be performed as according to Moore, et al., J. Clin. Microbiol., 36(4):1028-1031 (1998). SDA can be performed as according to Walker, et al., Nucleic Acids Res., 20(7):1691-1696 (1992). NASBA can be performed as according to Helm, et al., Nucleic Acids Res., 26(9):2250-2251 (1998). TMA can be performed as according to Wylie, et al., Journal of Clinical Microbiology, 36(12):3488-3491 (1998). All of the foregoing are incorporated by reference.

Polymerase Chain Reaction is described in R. K. Saiki et al., “Primer-directed Enzymatic Amplification of DNA With A Thermostable DNA Polymerase,” Science, pp. 487-491 (1988) (incorporated by reference). It is also described in Whelan, et al, J. Clin. Microbiol., 33(3):556-561 (1995) (incorporated by reference.)

Two currently available Taq polymerases for conducting PCR that are preferred for use with the present invention are Hot Start™ Taq polymerase from Quigen (Valencia, Calif.) and Jump Start Red™ Taq polymerase from Sigma-Aldrich (St. Louis, Mo.) These polymerases are usable pursuant to factory specifications. The factory specifications for Hot Start™ Taq is approximately as set out in the table below.

| Number of Cycles | Denaturation | Annealling | Polymerization |
|---|---|---|---|
| 1 | 94° or 95° C. for 15′ | | |
| 40 | 94° C. for 1′ | 58° C. for 1′ | 72° C. for 1′ |

′ = minutes
″ = seconds

It is preferable that the factory specifications for Hot Start™ Taq polymerase be modified after the first cycle. The factory specification of 40 cycles is broken down into 4 groups of 10 cycles. This allows for adjusting the time and temperature in each group. The reason for doing this is that in the first cycle, 100% of the templates are original chromosomes and fragments which are very long (1 billion bp.) In subsequent cycles, there is mixture of amplified targets from prior cycles and the original chromosome. The product PCR fragments are about 500 bp. Accordingly, the original chromosomes, or fragments, becomes less important.

The parameters for the first group of ten cycles is as set out in the table below.

| Number of | | | |
|---|---|---|---|
| Cycles | Denaturation | Annealling | Polymerization |
| 10 | 94° C. for 20″; The | 58° C. for 30″; The | 72° C. for 1′ |
| | time is lowered | time is decreased | |
| | from the factory 1′ | from the factory | |
| | to 20″ to reduce | specification of 1′ | |
| | enzyme death due | to 30″ to avoid | |
| | to heat. With less | non-specific (ns) | |
| | time there is less | annealing | |
| | damage to the | | |
| | enzyme. | | |

The parameters for the second group of ten cycles is as set out in the table below.

| | | | |
|---|---|---|---|
| Number of | | | |
| Cycles | Denaturation | Annealling | Polymerization |

| | | | |
|---|---|---|---|
| 10 | 94° C. for 15″; | 58° C. for 30″ | 72° C. for 3′; The |
| | Time is reduced | | time is increased |
| | from prior cycle of | | from the prior step |
| | 20″ and factory | | and factory |
| | specification of 1′ | | specification of 1′ |
| | because, to | | to 3′ becaue, (1) |
| | denature (i.e., | | the polymerase is |
| | make single | | dying because of |
| | stranded (ss)) | | higher |
| | template requires | | temperature and |
| | less time and/or a | | the effective |
| | lower temperature | | amount of |
| | than with a greater | | polymerase is less |
| | percentage of | | and (2) the |
| | longer templates. | | amount of |
| | | | fragment has |
| | | | increased |
| | | | exponentially |

The parameters for the third group of ten cycles is as set out in the table below.

| Number of Cycles | Denaturation | Annealling | Polymerization |
|---|---|---|---|
| 10 | 94° C. for 15″ | 58° C. for 30″ | 72° C. for 5′; The |
| | | | time is increased |
| | | | from the prior step |
| | | | of 3′ and factory |
| | | | specification of 1′ |
| | | | for same two |
| | | | reasons as set out |
| | | | above |

The parameters for the fourth group of ten cycles is as set out in the table below.

| Number of Cycles | Denaturation | Annealling | Polymerization |
|---|---|---|---|
| 10 | 94° C. for 15″ | 58° C. for 30″ | 72° C. for 7′; The |
| | | | time is increased |
| | | | from the prior step |
| | | | of 5′ and factory |
| | | | spec of 1′ to 3′ for |
| | | | same two reasons |
| | | | set out above |

The next step is detecting a target polynucleotide sequence. This is accomplished by methods well known to those skilled in the art; for, example Southern blotting, dot blotting and hybridization to DNA assays. A preferred method is described in F. Ausubel et al., “Current Protocols in Molecular Biology” (Wiley Interscience, N.Y.) (2000) (which is incorporated by reference).

Other aspects of the invention are kits for genotyping a animal by taking a tissue biopsy comprised of cells having polynucleotides along with other body components and detecting a target polynucleotide sequence through in situ Polymerase Chain Reaction without preliminary clean up steps. Interrelated aspects of the invention are kits for genotyping a mouse by taking a tail biopsy and detecting a target polynucleotide sequence through in situ Polymerase Chain Reaction without preliminary clean up steps.

The kits are comprised of a vial containing a lysing reagent as described above and one or more vials containing nucleic acid probes or primers complementary to the polynucleotide to be detected.

The previously described versions of the present invention have many advantages. One advantage is saving time. The table immediately below is a comparison between the Phenol/chloroform method, spooling method, DNeasy method and the present invention of the usual time expenditures (following a typical lab work up) for the steps after the lysis cycle and before the PCR step.

As stated in the Background Section, typically, there are 500 mice is a study. Accordingly, there are 500 tails. These tails are handled 50 at a time such that there are 10 groups of 50. The times in the table are the total for handling these 10 groups. That is, all the times in the table are 10× the time for one group of 50. For example, in the Phenol/chloroform method (column 1), doing the isolation step (row 2) for a group of 50 mice takes 6 hours. Since there are 10 groups, the total time is 60 hours.

For the spooling method (column 3), the resuspension time is 160 hours because, a pellet of stacked chromosomes is formed with strong Van der Wals and ionic electrostatic attraction between the stacked layers. Accordingly, it takes a long time to resuspend. For this invention (column 5), the heat treatment (row 4), time refers to the heat inactivation step. The 9.0 hour entry is generous. The steps could be done in 45 minutes per group of 10 for a total time of 7.5 hours.

| | Phenol/ | | | Present |
|---|---|---|---|---|
| | chloroform | Spooling | DNeasy | Invention |
| Time for DNA | 60 hours | 30 hours | 30 hours | O hours |
| Isolation | | | | |
| Time for | 160 hours | 160 hours | 0 hours | 0 hours |
| Resuspension | | | | |
| Time for Heat | 0 hours | O hours | 0 hours | 9.0 hours |
| Inactivation | | | | |
| Total Time | 220 hours | 190 hours | 30 hours | 9 hours |

Another advantage is saving money. The table immediately below is a comparison between the Phenol/chloroform method, spooling method, DNeasy method and the present invention of the usual costs (following a typical lab work up) for the steps after the lysis cycle and before the PCR.

Similarly to the prior table, the costs are stated for handling 500 mice (500 tails.) That is, all the costs 500× the costs of genotyping one mouse. The labor cost entry is based on an assumption of $12.00 per hour and the total time for the DNA isolation procedure of the prior art methods.

| | Phenol/ | | | Present |
|---|---|---|---|---|
| | chloroform | Spooling | DNeasy | Invention |
| 1.7 ml tubes | $48.00 | $48.00 | $24.00 | $0 |
| 200 1 tips | $14.00 | $42.00 | $14.00 | $0 |
| 1 ml tips | $44.00 | $44.00 | $14.00 | $0 |
| Phenol | $140.00 | $0 | $0 | $0 |
| Ethanol | $4.00 | $0 | $0 | $0 |
| Isopropanol | $0 | $4.00 | $0 | $0 |
| Proteinase K | $48.00 | $48.00 | $0 | $20.00 |
| Commercial Kit | $0 | $0 | $1054.00 | $0 |
| Labor | $720.00 | $360.00 | $180.00 | $0 |
| Total | $1018.00 | $546.00 | $2154.00 | $20.00 |

Other advantages of the present invention, include, but are not limited to, eliminating the use of phenol/chloroform which is dangerous to humans and the environment; minimizing the use of plasticware, such as tubes and tips, which are harmful to the environment; reducing the consumption of proteinase K; reducing loss of DNA from a tissue biopsy and reducing pain to rodents.

### EXAMPLE

The following example further describes and demonstrates embodiments within the scope of the present invention. The example is provided for the purpose of illustration and is not to be construed as limitations or restrictions of the present invention, as persons skilled in the art will quickly realize many variations thereof are possible that are all within the spirit and scope of the invention.

This example of the present invention illustrates the gender genotyping of mice using mouse tail biopsies. Specifically, tails (0.6 cm) were lysed in a lysing reagent containing 20 mM Tris-HCl, pH 8.5, 3 mM MgCl₂, 1 mM EDTA, 0.2% NP-40, 0.2% Triton X-100, 0.2% Tween-20, 100 mM NaCl, and 0.3 mg/ml proteinase K. To facilitate the lysising, the tubes containing tails were rotated in rotating hybridization oven. The complete lysis was achieved after 3 hours to over night. The crude lysates were heated at 85° C. for 45 minutes by floating the rack containing tubes in the water bath. The heated lysates were directly used for PCR.

The PCR was performed according to modified manufacturer's instruction manual (QIAGEN Product Guide, 1999). Specifically, the PCR reaction mixture (50 microliters) contained 1 microliter crude lysate, 10× reaction buffer, 0.2 mM dNTP mix (Boehringer Mannheim), 0.3 mM of each primer, and 1.25 units of Taq Polymerase (HotSar Taq polymerase (Quigen).) A Perkin Elmer Cycler Model 9600 was used. The PCR cycles were composed of:

1 cycle at 94° C. for 15 min;

followed by the sequential cycles (total 40 cycles) of

10 cycles at 94° C. for 20 seconds followed by 58° C. for 30 sec, then followed by 72° C. for 1 min,

6 cycles at 94° C. for 15 sec followed by 58° C. for 30 sec, then followed by 72° C. for 3 min,

6 cycles at 94° C. for 15 sec followed by 58° C. for 30 sec, then followed by 72° C. for 5 min,

6 cycles at 94° C. for 15 sec followed by 58° C. for 30 sec, then followed by 72° C. for 7 min,

6 cycles at 94° C. for 15 sec followed by 58° C. for 30 sec, then followed by 72° C. for 9 min,

6 cycles at 94° C. for 15 sec followed by 58° C. for 30 sec, then followed by 72° C. for 11 min.

Detection of amplified DNA fragments was conducted as follows: 15 microliters of PCR reaction were applied to 1% agarose-gel electrophoresis according to the method of Ausubel (F. Ausubel et al., “Current Protocols in Molecular Biology” (Wiley Interscience, N.Y.) (2000) (which is incorporated by reference).)

FIG. 1 is a photograph (image) of an electrophoresis gel illustrating the detection of a 618 base pair (bp) DNA fragment from the Y chromosome and a 221 bp DNA fragment from the X chromosome for gender determination of mice. The PCR primers were 5′-CCAACACTCTGCCTGCACCATTC (forward) and 5′-GGCTCGAGTTGTTTGCAGGCCCGC (reverse) specific for X-chromosome amplified 221 bp DNA fragment and primers 5′-TATCACTGTACTGAGTGTGATTAC-3′ (forward) and 5′-AGTTCTGAAGGCCTATGAAATC (reverse) specific for Y-chromosome amplified 618 bp DNA fragment. The markers were Low DNA Mass Ladder (Life Technologies).

Lanes 1 to 8 are various mice. Lane 9 is a control. The Control received the same PCR reaction using distilled water instead of crude lysates. The molecular weight markers are not shown. Male has XY sex chromosomes and female has XX sex chromosomes. The PCR reaction using the crude lysates prepared by this invention specifically amplified the expected 618 bp-DNA fragment from Y-chromosome, and the expected 221 bp-DNA fragment from X chromosome. In lanes 3 to 9 there is an unknown artifact at a mass slightly less than the 221 bp fragment which is inconsequential.

FIG. 2 is a photograph (image) of an electrophoresis gel illustrating the detection of a 409 base pair (bp) DNA fragment from the Y chromosome and the same 221 bp DNA fragment from the X chromosome for gender determination of mice. The Y-chromosome-specific primers 5′-TACAGTACCAACAAGAAGATAAGC (forward) and 5,-TTAGATTTTATATGAGTTTTCAAG (reverse) amplifying 409 bp DNA fragment.

Lanes 1 to 8 are various mice. Lane 9 is a control. The Control received the same PCR reaction using distilled water instead of crude lysates. The molecular weight markers are not shown. Male has XY sex chromosomes and female has XX sex chromosomes. The PCR reaction using the crude lysates prepared by this invention specifically amplified the expected 409 bp-DNA fragment from Y-chromosome, and the expected 221 bp-DNA fragment from X chromosome. This shows that this invention also works for another set of Y chromosome-specific primer pair that amplifies 409 bp, giving reproducible results.

Although the present invention has been described in considerable detail with reference to certain preferred versions thereof, other versions are possible with substituted, varied and/or modified materials and steps are employed. These other versions do not depart from the invention. Therefore, the spirit and scope of the appended claims should not be limited to the description of the preferred versions contained herein.

I claim:

1. A method for genotyping an animal comprising the steps of:
a. taking an animal tissue biopsy comprised of cells having polynucleotides along with other body components;
b. forming an admixture of the animal tissue biopsy with an effective amount of lysing reagent, the lysing reagent comprised of:
i. a combination of two or three non-ionic detergents selected from the group consisting of short chain octylphenoxy polyethoxy ethanol, medium chain octylphenoxy polyethoxy ethanol, long chain octylphenoxy polyethoxy ethanol, short chain polyoxyethlene sorbitan monolaurate, medium chain polyoxyethlene sorbitan monolaurate and long chain polyoxyethlene sorbitan monolaurate, each of the selected detergents at a concentration such that the combination is effective to facilitate the release of polynucleotides from cells in the animal tissue biopsy;
ii. a protease at a concentration that is effective to facilitate release of polynucleotides with the polynucleotides being in a condition that is sufficiently free from nucleic acid associated proteins such that a nucleic acid amplification step can be performed;
iii. a buffering agent which buffers the lysing reagent at a pH which is conducive to the functioning of both the protease and a polynucleotide polymerase to be added in an amplification step;
iv. a metal ion cofactor at a concentration that is effective to activate the protease and that is ineffective to significantly deactivate a polynucleotide polymerase to be added in an amplification step;
v. a chelating agent at a concentration that is effective to sufficiently inactivate attacking agents in the animal tissue biopsy and that is ineffective to significantly chelate the metal ion cofactor and
vi. a salt at a concentration effective to approximate physiological conditions for both the protease and a polynucleotide polymerase to be added in an amplification step;
c. heating the admixture for a period of time and under conditions effective to lyse a sufficient quantity of cells in the animal tissue biopsy, so as to form a crude lysate;
d. heating the crude lysate for a period of time and under conditions such that the protease and attacking agents in the animal tissue biopsy are significantly inactivated, so as to form a lysate;
e. amplifying in the lysate a target polynucleotide using a polynucleotide polymerase in an amplification method, without preliminary cleanup steps and
f. detecting the target polynucleotide sequence,
whereby a genotype of the animal is determined.

2. The method of claim 1 where the animal is a mouse and the animal tissue biopsy is taken from the mouse's tail.

3. The method of claim 2 where the polynucleotides are chromosomes or chromosome fragments and the target polynucleotide is a portion of a chromosome or chromosome fragment.

4. The method of claim 3 where the combination of nonionic detergents is NP40™ brand octylphenoxy polyethoxy ethanol sold by Sigma-Aldrich (St. Louis, Mo.), Triton X-100™ brand octylphenoxy polyethoxy ethanol sold by Rohm & Haas (Philadelphia, Pa.) and Tween-20™ brand polyoxyethlene sorbitan monolaurate sold by Imperial Chemical Industries Americas, Inc. (Bridgewater, N.J.), each at a nonzero concentration that sums to about 0.6% (v/v).

5. The method of claim 4 where the protease is between about 0.1 mg/ml to about 0.5 mg/ml proteinase K.

6. The method of claim 5 where the heating the admixture is for at least 1.2 hours at a temperature between about 50° C. to about 55° C. in a roller bed oven; the heating of the crude lysate is for at least about 30 minutes at a temperature between about 85° C. and less than about 90° C. in a hot water bath; the polynucleotide polymerase is Taq and the amplification method is PCR.

7. A method for genotyping a mouse comprising the steps of:
a. taking a biopsy of the mouse's tail such that cells having polynucleotides are removed along with other tail components;
b. forming an admixture of the tail biopsy with an effective amount of lysing reagent, the lysing reagent comprised of:
i. a combination of is NP40™ brand octylphenoxy polyethoxy ethanol sold by Sigma-Aldrich (St. Louis, Mo.), Triton X100™ brand octylphenoxy polyethoxy ethanol sold by Rohm & Haas (Philadelphia, Pa.) and Tween-20™ brand polyoxyethlene sorbitan monolaurate sold by Imperial Chemical Industries Americas, Inc. (Bridgewater, N.J.), each at a nonzero concentration that sums to about 0.6% (v/v);
ii. Proteinase K at a concentration between about 0.1 mg/ml to about 0.5 mg/ml;
iii. a buffering agent which buffers the lysing reagent at a pH between about 8.5 to about 8.8;
iv. a metal ion cofactor at a concentration between about 1 mM to about 5 mM;
v. a chelating agent at a concentration between about 0.5 mM to about 2 mM and
vi. a salt at a concentration between about 50 mM to about 200 mM;
c. heating the admixture for at least 1.2 hours at a temperature between about 50° C. to about 55° C., so as to form a crude lysate;
d. heating the crude lysate for about 30 minutes to about 180 minutes at a temperature between about 85° C. and less than about 90° C., so as to form a lysate;
e. amplifying in the lysate a target polynucleotide using Taq in a Polymerase Chain Reaction, without preliminary cleanup steps and
f. detecting the target polynucleotide,
whereby a genotype of the mouse is determined.

8. The method of claim 7 where the polynucleotides are chromosomes or chromosome fragments and the target polynucleotide is a portion of a chromosome or chromosome fragment.

9. The method of claim 8 where the combination of nonionic detergents is about 0.1% (v/v) NP40, about 0.1% Triton X-100 (v/v) and about 0.4% Tween-20 (v/v).

10. The method of claim 8 where the buffering agent is Tris-HCl; the metal ion cofactor is MgCl: the chelating agent is selected from the group consisting of ethylenediaminetetraacetic acid (EDTA) and ethyleneguaninetetraacetic acid (EGTA) and the salt is NaCl.

11. The method of claim 10 where the heating the admixture is in a roller bed oven and the heating of the crude lysate is in a shaker hot water bath.

12. A method for genotyping a mouse comprising the steps of:
a. taking a biopsy of the mouse's tail about 0.1 cm to about 0.6 cm in length such that cells having chromosomes are removed along with other tail components;
b. forming an admixture of the tail biopsy with about 40 micrometers to about 200 microliters of lysing reagent, the lysing reagent comprised of:
i. a combination of NP40™ brand octylphenoxy polyethoxy ethanol sold by Sigma-Aldrich (St. Louis, Mo.), Triton X-100™ brand octylphenoxy polyethoxy ethanol sold by Rohm & Haas (Philadelphia, Pa.) and Tween-20™ brand polyoxyethlene sorbitan monolaurate sold by Imperial Chemical Industries Americas, Inc. (Bridgewater, N.J.), each at a nonzero concentration that sums to about 0.6% (v/v);
ii. Proteinase K at a concentration of about 0.3 mg/ml;
iii. Tris-HCl buffering agent at about 80 mM so as to buffer the lysing reagent at a pH at about 8.5;
iv. MgCl₂ at a concentration of about 3 mM;
v. a chelating agent selected from the group of ethylenediaminetetraacetic acid (EDTA) and ethyleneguaninetetraacetic acid (EGTA) at a concentration of about 1.0 mM and
vi. NaCl at a concentration of about 150 mM;
c. heating the admixture for at least 1.2 hours at a temperature between about 50° C. to about 55° C. in a roller bed oven, so as to form a crude lysate;
d. heating the crude lysate for at least about 45 minutes at a temperature between about 85° C. and less than about 90° C. in hot water bath, so as to form a lysate;
e. amplifying in the lysate a target polynucleotide that is a portion of a chromosome or chromosome fragment using Taq in Polymerase Chain Reaction, without preliminary cleanup steps and
f. detecting the target polynucleotide,
whereby a genotype of the mouse is determined.

13. The method of claim 12 where the combination of nonionic detergents is about 0.1% (v/v) NP40, about 0.1% Triton X-100 (v/v) and about 0.4% Tween-20 (v/v).

14. The method of claim 12 where the heating the admixture is between about 4 to about 8 hours.

15. The method of claim 12 where the Polymerase Chain Reaction is carried out by a first denaturation cycle at about 94° C. for about 15 minutes; followed by about 10 cycles comprised of a denaturation at about 94° C. for about 20 seconds, an annealing at about 58° C. for about 30 seconds and a polymerization at 72° C. for about 1 minute; followed by about 10 cycles comprised of a denaturation at about 94° C. for about 15 seconds, an annealing at about 58° C. for about 30 seconds and a polymerization at 72° C. for about 3 minutes; followed by about 10 cycles comprised of a denaturation at about 94° C. for about 15 seconds, an annealing at about 58° C. for about 30 seconds and a polymerization at 72° C. for about 5 minutes and followed by about 10 cycles comprised of a denaturation at about 94° C. for about 15 seconds, an annealing at about 58° C. for about 30 seconds and a polymerization at 72° C. for about 7 minutes.

16. The method of claim 13 where the Polymerase Chain Reaction is carried out by a first denaturation cycle at about 94° C. for about 15 minutes; followed by about 10 cycles comprised of a denaturation at about 94° C. for about 20 seconds, an annealing at about 58° C. for about 30 seconds and a polymerization at 72° C. for about 1 minute; followed by about 10 cycles comprised of a denaturation at about 94° C. for about 15 seconds, an annealing at about 58° C. for about 30 seconds and a polymerization at 72° C. for about 3 minutes; followed by about 10 cycles comprised of a denaturation at about 94° C. for about 15 seconds, an annealing at about 58° C. for about 30 seconds and a polymerization at 72° C. for about 5 minutes and followed by about 10 cycles comprised of a denaturation at about 94° C. for about 15 seconds, an annealing at about 58° C. for about 30 seconds and a polymerization at 72° C. for about 7 minutes.

17. A composition for releasing polynucleotides from an animal tissue biopsy comprised of cells having polynucleotides along with other body components in a form suitable for in situ Polymerase Chain Reaction of a target polynucleotide without preliminary clean up steps comprising:
a. a combination of two or three non-ionic detergents selected from the group consisting of short chain octylphenoxy polyethoxy ethanol, medium chain octylphenoxy polyethoxy ethanol, long chain octylphenoxy polyethoxy ethanol, short chain polyoxyethlene sorbitan monolaurate, medium chain polyoxyethlene sorbitan monolaurate and long chain polyoxyethlene sorbitan monolaurate, each of the selected detergents at a concentration such that the combination is effective to facilitate the release of polynucleotides from cells in the animal tissue biopsy;
b. a protease at a concentration that is effective to facilitate the release of polynucleotides from the cells with the polynucleotides being in a condition that is sufficiently free from nucleic acid associated proteins such that a nucleic acid amplification step can be performed;
c. a buffering agent which buffers the lysing reagent at a pH which is conducive to the functioning of both the protease and a polynucleotide polymerase to be added in an amplification step;
d. a metal ion cofactor at a concentration that is effective to activate the protease and that is ineffective to significantly deactivate a polynucleotide polymerase to be added in an amplification step;
e. a chelating agent at a concentration that is effective to sufficiently inactivate attacking agents in the tail biopsy and that is ineffective to significantly chelate the metal ion cofactor and
f. a salt at a concentration effective to approximate physiological conditions for both the protease and a polynucleotide polymerase to be added in an amplification step.

18. The composition of claim 17 where the animal is a mouse and the animal tissue biopsy is taken from the mouse's tail.

19. The composition of claim 18 where the polynucleotides are chromosomes or chromosome fragments and the target polynucleotide is a portion of a chromosome or chromosome fragment.

20. The composition of claim 19 where the combination of nonionic detergents is is NP40™ brand octylphenoxy polyethoxy ethanol sold by Sigma-Aldrich (St. Louis, Mo.), Triton X-10™ brand octylphenoxy polyethoxy ethanol sold by Rohm & Haas (Philadelphia, Pa.) and Tween-20™ brand polyoxyethlene sorbitan monolaurate sold by Imperial Chemical Industries Americas, Inc. (Bridgewater, N.J.), each at a nonzero concentration that sums to about 0.6% (v/v).

21. The composition of claim 20 where the protease is between about 0.1 mg/ml to about 0.5 mg/ml proteinase K.

22. A composition for releasing polynucleotides from a mouse tail biopsy comprised of cells having polynucleotides along with other body components in a form suitable for in situ Polymerase Chain Reaction of a target polynucleotide without preliminary clean up steps comprising:
a. a combination of NP40™ brand octylphenoxy polyethoxy ethanol sold by Sigma-Aldrich (St. Louis, Mo.), Triton X-100™ brand octylphenoxy polyethoxy ethanol sold by Rohm & Haas (Philadelphia, Pa.) and Tween-20™ brand polyoxyethlene sorbitan monolaurate sold by Imperial Chemical Industries Americas, Inc. (Bridgewater, N.J.), each at a nonzero concentration that sums to about 0.6% (v/v);
b. Proteinase K at a concentration between about 0.1 mg/ml to about 0.5 mg/ml;
c. a buffering agent which buffers the lysing reagent at a pH between about 8.5 to about 8.8;
d. a metal ion cofactor at a concentration between about 1 mM to about 5 mM;
e. a chelating agent at a concentration between about 0.5 mM to about 2 mM and
f. a salt at a concentration between about 50 mM to about 200 mM.

23. The composition of claim 22 where the polynucleotides are chromosomes or chromosome fragments and the target polynucleotide is a portion of a chromosome or chromosome fragment.

24. The composition of claim 23 where the combination of nonionic detergents is about 0.1% (v/v) NP40, about 0.1% Triton X-100 (v/v) and about 0.4% Tween-20 (v/v).

25. The composition of claim 23 where the buffering agent is Tris-HCl; the metal ion cofactor is MgCl; the chelating agent is selected from the group consisting of ethylenediaminetetraacetic acid (EDTA) and ethyleneguaninetetraacetic acid (EGTA) and the salt is NaCl.

26. A composition for releasing chromosomes or chromosome fragments from a mouse tail biopsy comprised of cells having chromosomes along with other body components in a form suitable for in situ Polymerase Chain Reaction of a target polynucleotide which is a portion of a chromosome or chromosome fragment without preliminary clean up steps comprising:
a. a combination of NP40™ brand octylphenoxy polyethoxy ethanol sold by Sigma-Aldrich (St. Louis, Mo.), Triton X-100™ brand octylphenoxy polyethoxy ethanol sold by Rohm & Haas (Philadelphia, Pa.) and Tween-20™ brand polyoxyethlene sorbitan monolaurate sold by Imperial Chemical Industries Americas, Inc. (Bridgewater, N.J.), each at a nonzero concentration that sums to about 0.6% (v/v);
b. Proteinase K at a concentration of about 0.3 mg/ml;
c. Tris-HCl buffering agent at about 80 mM so as to buffer the lysing reagent at a pH at about 8.5;
d. MgCl₂ at a concentration of about 3 mM;
e. a chelating agent selected from the group of ethylenediaminetetraacetic acid (EDTA) at a concentration of about 1.0 mM and
f. NaCl at a concentration of about 150 mM.

27. The composition of claim 26 where the combination of nonionic detergents is about 0.1% (v/v) NP40, about 0.1% Triton X-100 (v/v) and about 0.4% Tween-20 (v/v).

28. A kit for genotyping a animal by taking a biopsy comprised of cells having polynucleotides along with other body components and detecting a target polynucleotide sequence through in situ Polymerase Chain Reaction without preliminary clean up steps comprised of:
a. a vial containing the composition of claim 17 and
b. one or more vials containing nucleic acid probes or primers complementary to the polynucleotide sequence to be detected.

29. A kit for genotyping a mouse by taking a tail biopsy comprised of cells having polynucleotides along with other body components and detecting a target polynucleotide sequence through in situ Polymerase Chain Reaction without preliminary clean up steps comprised of:
a. a vial containing the composition of claim 22 and
b. one or more vials containing nucleic acid probes or primers complementary to the polynucleotide sequence to be detected.

30. A kit for genotyping a mouse by taking a tail biopsy comprised of cells having chromosomes or chromosome fragments along with other body components and detecting a target polynucleotide sequence which is a portion of the chromosome or chromosome fragment through in situ Polymerase Chain Reaction without preliminary clean up steps comprised of:
a. a vial containing the composition of claim 26 and
b. one or more vials containing nucleic acid probes or primers complementary to the polynucleotide sequence to be detected.
